SEQUENCE LISTING (16 sequences)

SEQ ID NO: 1 - DNA, 252 bp, Corynebacterium glutamicum
atggtccacaacggcatcgagtacgccgacatgcaggtcatcggcgaggcataccaccttctgccctacgcagcaggcatgcagccagctgaaatcgctgaggttttcaaggaatggaacgcaggcgacctggattcctacctcatcgaaatcaccgcagaggttctctcccaggtggatgctgaaaccggcaagccactaatcgacgtcatcgttgacgctgcaggtcagaagggcaccggcaagtggact

SEQ ID NO: 2 - DNA, 2335 bp, Corynebacterium glutamicum
  CDS (474)..(1850): gnd
ttgttcggccacgatgacaccggagctcacagcagaaatgaagtcggtgttgttgttgatgccgacgaccatttttccaggggcggaaatcatgctggcgactgatccagtggattcggcgatggcggcgtagacaccaccgttgaccaagcccaccacttgcaggtgcttggatgccacgtgaagttcgctgaccacccggccgggctcgatggtggtgtagcgcagccccagattgcggtcgaggccataattggcgttgttgagtgcttcaagttcgtctgtggttaaagctctggtggcggcaagttctgcaagcgaaagcagatcttggggttgatcatcgcgggaagtcataattaattactctagtcggcctaaaatggttggattttcacctcctgtgacctggtaaaatcgccactacccccaaatggtcacaccttttaggccgattttgctgacaccgggctatgccgtcaagtacgatcaataacatgactaatggagataatctcgcacagatcggcgttgtaggcctagcagtaatgggctcaaacctcgcccgcaacttcgcccgcaacggcaacactgtcgctgtctacaaccgcagcactgacaaaaccgacaagctcatcgccgatcacggctccgaaggcaacttcatcccttctgcaaccgtcgaagagttcgtagcatccctggaaaagccacgccgcgccatcatcatggttcaggctggtaacgccaccgacgcagtcatcaaccagctggcagatgccatggacgaaggcgacatcatcatcgacggcggcaacgccctctacaccgacaccattcgtcgcgagaaggaaatctccgcacgcggtctccacttcgtcggtgctggtatctccggcggcgaagaaggcgcactcaacggcccatccatcatgcctggtggcccagcaaagtcctacgagtccctcggaccactgcttgagtccatcgctgccaacgttgacggcaccccatgtgtcacccacatcggcccagacggcgccggccacttcgtcaagatggtccacaacggcatcgagtacgccgacatgcaggtcatcggcgaggcataccaccttctgccctacgcagcaggcatgcagccagctgaaatcgctgaggttttcaaggaatggaacgcaggcgacctggattcctacctcatcgaaatcaccgcagaggttctctcccaggtggatgctgaaaccggcaagccactaatcgacgtcatcgttgacgctgcaggtcagaagggcaccggcaagtggactgtcaaggctgctcttgatctgggtattgctaccaccggcatcggcgaacgtgttttcgcacgtgcactctccggcgcaaccagccagcgcgctgcagcacagggcaacctacctgcaggtgtcctcaccgatctggaagcacttggcgtggacaaggcacagttcgtcgaaggacttcgccgtgcactgtacgcatccaagcttgttgcttacgcacagggcttcgacgagatcaaggctggctccgacgagaacaactgggacgttgaccctcgcgacctcgctaccatctggcgcggcggctgcatcattcgcgctaagttcctcaaccgcatcgtcgaagcatacgatgcaaacgctgaacttgagtccctgctgctcgatccttacttcaagagcgagctcggcgacctcatcgattcatggcgtcgcgtgattgtcaccgccacccagcttggcctgccaatcccagtgttcgcttcctccctgtcctactacgacagcctgcgtgcagagcgtctgccagcagccctgatccactagtgtcgacctgcaggcgcgcgagctccagcttttgttccctttagtgagggttaatttcgagcttggcgtaatcaaggtcatagctgtttcctgtgtgaaattgttatccgctcacaattccacacaatatacgagccggaagtataaagtgtaaagcctggggtgcctaatgagtgagctaactcacagtaattgcggctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtccc

SEQ ID NO: 3 - protein, 459 aa, Corynebacterium glutamicum
MPSSTINNMTNGDNLAQIGVVGLAVMGSNLARNFARNGNTVAVYNRSTDKTDKLIADHGSEGNFIPSATVEEFVASLEKPRRAIIMVQAGNATDAVINQLADAMDEGDIIIDGGNALYTDTIRREKEISARGLHFVGAGISGGEEGALNGPSIMPGGPAKSYESLGPLLESIAANVDGTPCVTHIGPDGAGHFVKMVHNGIEYADMQVIGEAYHLLPYAAGMQPAEIAEVFKEWNAGDLDSYLIEITAEVLSQVDAETGKPLIDVIVDAAGQKGTGKWTVKAALDLGIATTGIGERVFARALSGATSQRAAAQGNLPAGVLTDLEALGVDKAQFVEGLRRALYASKLVAYAQGFDEIKAGSDENNWDVDPRDLATIWRGGCIIRAKFLNRIVEAYDANAELESLLLDPYFKSELGDLIDSWRRVIVTATQLGLPIPVFASSLSYYDSLRAERLPAALIH

SEQ ID NO: 4 - DNA, 2160 bp, Corynebacterium glutamicum
  CDS (327)..(2063): poxB
ttagaggcgattctgtgaggtcactttttgtggggtcggggtctaaatttggccagttttcgaggcgaccagacaggcgtgcccacgatgtttaaataggcgatcggtgggcatctgtgtttggtttcgacgggctgaaaccaaaccagactgcccagcaacgacggaaatcccaaaagtgggcatccctgtttggtaccgagtacccacccgggcctgaaactccctggcaggcgggcgaagcgtggcaacaactggaatttaagagcacaattgaagtcgcaccaagttaggcaacacaatagccataacgttgaggagttcagatggcacacagctacgcagaacaattaattgacactttggaagctcaaggtgtgaagcgaatttatggtttggtgggtgacagccttaatccgatcgtggatgctgtccgccaatcagatattgagtgggtgcacgttcgaaatgaggaagcggcggcgtttgcagccggtgcggaatcgttgatcactggggagctggcagtatgtgctgcttcttgtggtcctggaaacacacacctgattcagggtctttatgattcgcatcgaaatggtgcgaaggtgttggccatcgctagccatattccgagtgcccagattggttcgacgttcttccaggaaacgcatccggagattttgtttaaggaatgctctggttactgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgcgattggtgcgcaaagtgttgatcgaaaccgccaggtgatcgcgatgtgtggcgatggtggtttgggcatgctgctgggtgagcttctgaccgttaagctgcaccaacttccgctgaaggctgtggtgtttaacaacagttctttgggcatggtgaagttggagatgctcgtggagggacagccagaatttggtactgaccatgaggaagtgaatttcgcagagattgcggcggctgcgggtatcaaatcggtacgcatcaccgatccgaagaaagttcgcgagcagctagctgaggcattggcatatcctggacctgtactgatcgatatcgtcacggatcctaatgcgctgtcgatcccaccaaccatcacgtgggaacaggtcatgggattcagcaaggcggccacccgaaccgtctttggtggaggagtaggagcgatgatcgatctggcccgttcgaacataaggaatattcctactccatgatgattgatacacctgctgttctcattgaccgcgagcgcttaactgccaacatttccaggatggcagctcacgccggtgcccatgagattgccct

SEQ ID NO: 5 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 6 - DNA, 875 bp, Corynebacterium glutamicum
tgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgc

SEQ ID NO: 7 - DNA, 23 bp, Artificial sequence
  Description of artificial sequence Primer gnd1
atggtkcacacyggyatygarta

SEQ ID NO: 8 - DNA, 21 bp, Artificial sequence
  Description of artificial sequence Primer gnd2
rgtccayttrccrgtrccytt

SEQ ID NO: 9 - DNA, 17 bp, Artificial sequence
  Description of artificial sequence Internal primer 1
ggtggatgctgaaaccg

SEQ ID NO: 10 - DNA, 17 bp, Artificial sequence
  Description of artificial sequence Internal primer 2
gctgcatgcctgctgcg

SEQ ID NO: 11 - DNA, 17 bp, Artificial sequence
  Description of artificial sequence  Internal primer 3
ttgttgcttacgcacag

SEQ ID NO: 12 - DNA, 17 bp, Artificial sequence
  Description of artificial sequence Internal primer 4
tcgtaggactttgctgg

SEQ ID NO: 13 - DNA, 21 bp, Artificial sequence
  Description of artificial sequence gnd fwd. primer
actctagtcggcctaaaatgg

SEQ ID NO: 14 - DNA, 21 bp, Artificial sequence
  Description of artificial sequence gnd rev. primer
cacacaggaaacagatatgac

SEQ ID NO: 15 - DNA, 20 bp, Artificial sequence
  Description of artificial sequence Primer poxBint1
tgcgagatggtgaatggtgg

SEQ ID NO: 16 - DNA, 20 bp, Artificial sequence
  Description of artificial sequence Primer poxBint2
gcatgaggcaacgcattagc

(19) United States
(12) Patent Application Publication
(10) Pub. No.: US 2003/0119154 A1
(43) Pub. Date: Jun. 26, 2003

(54) Process for the preparation of L-amino acids using a gene encoding 6-phosphogluconate dehydrogenase

(76) Inventors: L. K. Dunican, Galway (IE); Ashling McCormack, Athlone (IE); Cliona Stapelton, Roscrea (IE); Kevin Burke, Galway (IE); Bettina Mockel, Bielefeld (DE)

Correspondence Address: PILLSBURY WINTHROP, LLP, P.O. BOX 10500, MCLEAN, VA, 22102, US

(21) Appl. No.: 10/078,167

(22) Filed: Feb. 20, 2002

Related U.S. Application Data
(63) Continuation-in-part of application No. 09/531,265, filed on Mar. 20, 2000, now abandoned.

(51) Int. Cl. C12P 13/08; C07H 21/04; C12N 9/02; C12P 21/02; C12N 1/21; C12N 15/74

(52) U.S. Cl. 435/115; 435/69.1; 435/252.3; 435/320.1; 435/189; 536/23.2

(57) ABSTRACT

The invention relates to a process for the preparation of L-amino acids. The process involves fermenting an L-amino acid producing coryneform bacteria in a culture medium, concentrating L-amino acid produced by the fermenting in the culture medium or in the cells of the bacteria, and isolating the L-amino acid produced. The bacteria has an overexpressed gene encoding 6-phosphogluconate dehydrogenase and a decreased or switched off gene encoding pyruvate oxidase. The L-amino acid may be L-lysine, L-threonine, L-isoleucine or L-tryptophan.

### CROSS REFERENCE TO RELATED APPLICATIONS

The present application is a continuation-in-part of U.S. application Ser. No. 09/531,265, filed on Mar. 20, 2000, the contents of which are incorporated by reference herein in their entirety.

### FIELD OF THE INVENTION

The invention relates to a process for the fermentative preparation of L-amino acids, in particular L-lysine, L-threonine, L-isoleucine and L-tryptophan, using coryneform bacteria in which at least the enzyme 6-phosphogluconate dehydrogenase encoded by the gnd gene is amplified.

### BACKGROUND

L-Amino acids are used in animal nutrition, in human medicine and in the pharmaceuticals industry and are prepared by fermentation from strains of coryneform bacteria, in particular Corynebacterium glutamicum. Because of their great importance, work is constantly being undertaken to improve the preparation processes. Improvements may relate to fermentation measures, e.g., stirring and supply of oxygen; the composition of the nutrient media, e.g., the sugar concentration during the fermentation; the working up to the product form, e.g., by ion exchange chromatography; or the intrinsic output properties of the microorganism itself.

Methods of mutagenesis, selection and mutant selection are used to improve the output properties of these microorganisms. Strains which are resistant to antimetabolites (e.g., the threonine analogue α-amino-β-hydroxyvaleric acid (AHV), and the lysine analogue S-(2-aminoethyl)-L-cystein (AEC)) or which are auxotrophic for metabolites of regulatory importance and produce L-amino acids such as threonine or lysine are obtained in this manner.

Methods utilizing recombinant DNA techniques have also been employed for some years for improving Corynebacterium glutamicum strains which produce L-amino acids.

### SUMMARY OF THE INVENTION

L-Amino acids are used in human medicine and in the pharmaceuticals industry, in the foodstuffs industry and especially in animal nutrition. There is therefore a general interest in providing improved processes for their preparation.

In general, the present invention is directed to improved processes for the fermentative preparation of L-amino acids by coryneform bacteria. More specifically, the invention provides a process for the fermentative preparation of L-amino acids (particularly L-lysine, L-threonine, L-isoleucine and L-tryptophan) using coryneform bacteria in which the nucleotide sequence which codes for the enzyme 6-phosphogluconate dehydrogenase (EC number 1.1.1.44) (gnd gene) is amplified, in particular over-expressed.

### BRIEF DESCRIPTION OF THE FIGURES

Embodiments of the present invention will be described with reference to the following Figures, in which:

FIG. 1 is a map of the plasmid pEC-Ti 18mob2;

FIG. 2 is a map of the plasmid pECgnd;

FIG. 3 is a map of the plasmid pBGNA; and

FIG. 4 is a map of the plasmid pCR2. 1poxBint.

### DETAILED DESCRIPTION OF THE INVENTION

The strains of bacteria employed in the present processes preferably already produce L-amino acids before amplification of the gnd gene. The term “amplification” as used herein describes the increase in the intracellular activity of one or more enzymes or proteins in a microorganism which are encoded by the corresponding DNA. This may be accomplished, for example, by increasing the number of copies of the gene or genes, using a potent promoter or using a gene which codes for a corresponding enzyme having a high activity, or by combining these measures.

By amplification measures, in particular over-expression, the activity or concentration of the corresponding enzyme or protein is in general increased by at least 10%, 25%, 50%, 75%, 100%, 150%, 200%, 300%, 400% or 500%, up to a maximum of 1000% or 2000%, compared to that of the wild-type enzyme or the activity or concentration of the enzyme in the starting microorganism.

The microorganisms which the present invention provide can prepare L-aminio acids from glucose, sucrose, lactose, fructose, maltose, molasses, starch, cellulose or from glycerol and ethanol. They are representatives of coryneform bacteria, in particular of the genus Corynebacterium. Of the genus Corynebacterium, the most preferred species is Corynebacterium glutamicum, which is known among experts for its ability to produce L-amino acids. Suitable strains include the wild-type strains:

Corynebacteruim glutamicum ATCC13032;

Corynebacterium acetoglutamicum ATCC15806;

Corynebacterium acetoacidophilum ATCC13870;

Corynebacterium thermoaminogenes FERM BP-1539;

Brevibacterium flavum ATCC14067;

Brevibacterium lactofermentum ATCC13869;

Brevibacterium divaricatum ATCC14020;

L-amino acid-producing mutants prepared from the strains above may also be used. f Such strains include: the L-threonine-producing strains:

Corynebacteruim glutamicum ATCC2 1649;

Brevibacterium flavum BB69;

Brevibacterium flavum DSM5399;

Brevibacterium lactofermentum FERM-BP 269;

Brevibacterium lactofermentum TBB-10;

the L-isoleucine-producing strains:

Corynebacteruim glutamicum ATCC 14309;

Corynebacteruim glutamicum ATCC 14310;

Corynebacteruim glutamicum ATCC 14311;

Corynebacteruim glutamicum ATCC 15168;

Corynebacterium ammoniagenes ATCC 6871;

the L-tryptophan-producing strains:

Corynebacteruim glutamicum ATCC21850;

Corynebacteruim glutamicum KY9218(pKW9901);

and the L-lysine-producing strains:

Corynebacteruim glutamicum FERM-P 1709;

Brevibacterium flavum FERM-P 1708;

Brevibacterium lactofermentum FERM-P 1712;

Corynebacteruim glutamicum FERM-P 6463;

Corynebacteruim glutamicum FERM-P 6464;

Corynebacteruim glutamicum DSM5715;

Corynebacteruim glutamicum DM58-1; and

Corynebacteruim glutamicum DSM12866.

It has been found that coryneform bacteria produce L-amino acids, in particular L-lysine, L-threonine, L-isoleucine and L-tryptophan, in an improved manner after over-expression of the gnd gene. The gnd gene codes for the enzyme 6-phosphogluconate dehydrogenase (EC number 1.1.1.44) which catalyses the oxidative decarboxylation of 6-phosphogluconic acid to ribulose 5-phosphate. The nucleotide sequence of the gnd gene is disclosed in JP-A-9-224662. Alleles of the gnd gene which result from the degeneracy of the genetic code or which are due to sense mutations of neutral function can furthermore be used. Genes encoding proteins with 6-phosphogluconate dehydrogenase activity from Gram-negative bacteria, e.g. Escherichia coli, or other Gram-positive bacteria, e.g., Streptomyces or Bacillus, may optionally be used.

The use of endogenous, genes in particular endogenous genes from coryneform bacteria, is preferred. The terms “endogenous genes” or “endogenous nucleotide sequences” refer to genes or nucleotide sequences which are available in the population of a species.

To achieve an amplification (e.g., over-expression) of a protein, the number of copies of the corresponding gene is increased, or the promoter and regulation region or the ribosome binding site upstream of the structural gene are mutated. Expression cassettes which are incorporated upstream of the structural gene act in the same way. Using inducible promoters, it is additionally possible to increase the expression in the course of fermentative L-amino acid formation. Expression may also be improved by measures to prolong the life of the m-RNA. Enzyme activity may be increased by preventing the degradation of the enzyme protein.

Genes or gene constructs may either be provided in plasmids with a varying number of copies, or may be integrated and amplified in the chromosome. Alternatively, an over-expression of the genes in question can be achieved by changing the composition of the media and the culture procedure. Instructions in this context can be found by the expert, inter alia, in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European Patent Specification EPS 0 472 869, in U.S. Pat. No. 4,601,893, in Schwarzer and Puhler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in Patent Application WO 96/15246, in Malumbres et al. (Gene 134, 15-24 (1993)), in Japanese Laid-Open Specification JP-A-10-229891, in Jensen and Hammer (Biotechnology and Bioengineering 58, 191-195 (1998)) and in known textbooks of genetics and molecular biology.

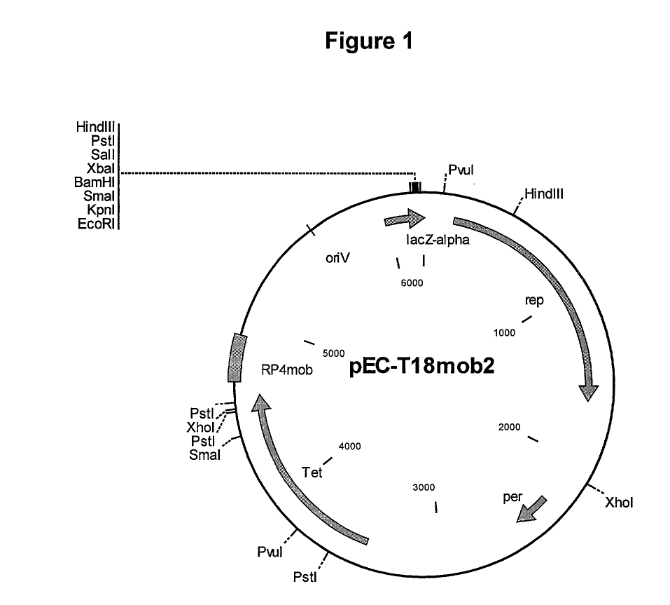

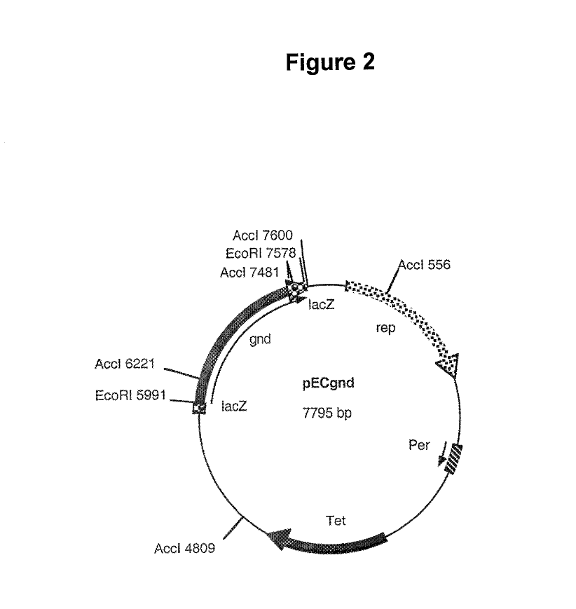

By way of example, 6-phosphogluconate dehydrogenase was over-expressed with the aid of a plasmid. The E. coli—C. glutamicum shuttle vector pEC-T18mob2 shown in FIG. 1 was used for this. After incorporation of the gnd gene into the EcoRI cleavage site of pEC-T 1 8mob2, the plasmid pECgnd shown in FIG. 2 was formed. Other plasmid vectors which are capable of replication in C. glutamicum, such as pEKEx1 (Eikmanns et al., Gene 102:93-98 (1991)) or pZ8-1 (EP-B-0 375 889), can be used in the same way.

In addition, it may be advantageous for the production of L-amino acids to amplify one or more enzymes of the relevant biosynthesis pathway, of glycolysis, of anaplerosis, of the pentose phosphate pathway or of amino acid export, in addition to amplification of the gnd gene. For example, for the preparation of L-threonine, one or more of the following genes can be amplified (over-expressed):

the hom gene which codes for homoserine dehydrogenase (Peoples et al., Molecular Microbiology 2, 63-72 (1988)) or the hom^{dr} allele which codes for a “feed back resistant” homoserine dehydrogenase (Archer et al., Gene 107, 53-59 (1991),

the gap gene which codes for glyceraldehyde 3-phosphate dehydrogenase (Eikmanns et al., Journal of Bacteriology 174: 6076-6086 (1992)),

the pyc gene which codes for pyruvate carboxylase (Peters-Wendisch et al., Microbiology 144: 915-927 (1998)),

the mqo gene which codes for malate:quinone oxidoreductase (Molenaar et al., European Journal of Biochemistry 254, 395-403 (1998)),

the tkt gene which codes for transketolase (accession number AB023377 of the databank of European Molecular Biology Laboratories (EMBL, Heidelberg, Germany)),

the zwf gene which codes for glucose 6-phosphate dehydrogenase (JP-A-09224661),

the thrE gene which codes for threonine export (DE 199 41 478.5; DSM 12840),

the zwal gene (DE 199 59 328.0; DSM 13115),

the eno gene which codes for enolase (DE: 199 41 478.5).

For the preparation of L-lysine, one or more of the following genes can be amplified, in particular over-expressed, at the same time as gnd.

the dapA gene which codes for dihydrodipicolinate synthase (EP-B 0 197 335),

a lysC gene which codes for a feed back resistant aspartate kinase (Kalinowski et al. (1990), Molecular and General Genetics 224: 317-324),

the gap gene which codes for glyceraldehyde 3-phosphate dehydrogenase (Eikmanns (1992), Journal of Bacteriology 174:6076-6086),

the pyc gene which codes for pyruvate carboxylase (Eikmanns (1992), Journal of Bacteriology 174:6076-6086),

the mqo gene which codes for malate-quinone oxidoreductase (Molenaar et al., European Journal of Biochemistry 254, 395-403 (1998)),

the tkt gene which codes for transketolase (accession number AB023377 of the databank of European Molecular Biologies Laboratories (EMBL, Heidelberg, Germany)),

the zwf gene which codes for glucose 6-phosphate dehydrogenase (JP-A-09224661),

the lysE gene which codes for lysine export (DE-A-195 48 222),

the zwa1 gene (DE 199 59 328.0; DSM 13115),

the eno gene which codes for enolase (DE 199 47 791.4).

The use of endogenous genes is preferred.

It may furthermore be advantageous for the production of L-amino acids to attenuate one or more of the following genes while at the same time amplifying gnd: the pck gene which codes for phosphoenol pyruvate carboxykinase (DE 199 50 409.1; DSM 13047),

the pgi gene which codes for glucose 6-phosphate isomerase (U.S. Ser. No. 09/396,478, DSM 12969),

the poxB gene which codes for pyruvate oxidase (DE 199 51 975.7; DSM 13114),

the zwa2 gene (DE: 199 59 327.2; DSM 13113).

In this connection, the term “attenuation” means reducing or suppressing the intracellular activity or concentration of one or more enzymes or proteins in a microorganism. This may be accomplished using the genes which encode the proteins, for example by using a weak promoter or a gene or allele which codes for a corresponding protein which has a low activity or inactivates the corresponding enzyme and optionally by combining these measures. By attenuation measures, the activity or concentration of the corresponding enzyme or protein is in general reduced to 0 to 75%, 0 to 50%, 0 to 25%, 0 to 10% or 0 to 5% of the activity or concentration of the wild-type enzyme or of the activity or concentration of the enzyme in the starting microorganism.

In addition to over-expression of 6-phosphogluconate dehydrogenase, it may furthermore be advantageous for the production of L-amino acids to eliminate undesirable side reactions (see, Nakayama: “Breeding of Amino Acid Producing Micro-organisms organisms,” in: Overproduction of Microbial Products, Krumphanzl, Sikyta, Vanek (eds.), Academic Press, London, UK, 1982).

The microorganisms prepared according to the invention can be cultured continuously or discontinuously in a batch process (batch culture) or in a fed batch (feed process) or repeated fed batch process (repetitive feed process) for the purpose of L-amino acid production. A summary of known culture methods is described in the textbook by Chmiel (Bioprozesstechnik 1. Einfuhrung in die Bioverfahrenstechnik [Bioprocess Technology 1. Introduction to Bioprocess Technology (Gustav Fischer Verlag, Stuttgart, 1991)] or in the textbook by Storhas (Bioreaktoren und periphere Einrichtungen [Bioreactors and Peripheral Equipment] (Vieweg Verlag, Braunschweig/Wiesbaden, 1994)). The culture medium to be used must meet the requirements of the particular microorganisms in a suitable manner. Descriptions of culture media for various microorganisms are contained in the handbook “Manual of Methods for General Bacteriology” of the American Society for Bacteriology (Washington D.C., USA, 1981). Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture. Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture. Potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia, or acid compounds, such as phosphoric acid or sulfuric acid, can be employed in a suitable manner to control the pH. Antifoams, such as fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of L-amino acid has formed. This target is usually reached within 10 hours to 160 hours.

The analysis of L-amino acids can be carried out by anion exchange chromatography with subsequent ninhydrin derivation, as described by Spackman et al. (Analytical Chemistry, 30, (1958), 1190), or it can take place by reversed phase HPLC as described by Lindroth et al. (Analytical Chemistry (1979) 51:. 1167-1174).

The following microorganism has been deposited at the Deutsche Sammlung fu ar Mikroorganismen und Zellkulturen (DSMZ=German Collection of Microorganisms and Cell Cultures, Braunschweig, Germany) in accordance with the Budapest Treaty: Escherichia coli K-12 DH5α/pEC-T18mob2 as DSM 13244.

In the accompanying Figures, the base pair numbers stated are approx. values obtained in the context of reproducibility. The abbreviations used in the Figures have the following meaning:

In FIG. 1:

| | |
|---|---|
| Tet: | Resistance gene for tetracycline |
| oriV: | Plasmid-coded replication origin of E. coli |
| RP4mob: | mob region for mobilizing the plasmid |
| rep: | Plasmid-coded replication origin from C. glutamicum |
| | plasmid pGA1 |
| per: | Gene for controlling the number of copies from pGA1 |
| lacZ-alpha: | lacZα gene fragment (N-terminus) |
| | of the β-Galactosidase gene. |

In FIG. 2:

| | |
|---|---|
| Tet: | Resistance gene for tetracycline |
| rep: | Plasmid-coded replication origin from C. glutamicum |
| | plasmid pGA1 |
| per: | Gene for controlling the number of copies from PGA1 |
| lacZ | Cloning relict of the lacZα gene fragment |
| | from pEC-T18mob2 |
| gnd: | 6-Phosphogluconate dehydrogenase gene. |

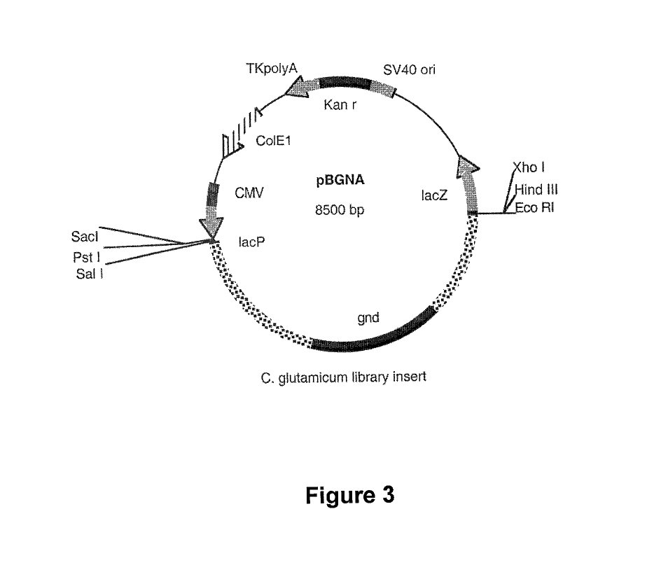

In FIG. 3:

| | |
|---|---|
| LacP: | Promoter of the E. coli lactose operon |
| CMV: | Promoter of cytomegalovirus |
| ColE1: | Replication origin of the plasmid ColE1 |
| TkpolyA: | Polyadenylation site |
| Kan r: | Kanamycin resistance gene |
| SV40ori: | Replication origin of Simian virus 40 |
| gnd: | 6-Phosphogluconate dehydrogenase gene. |

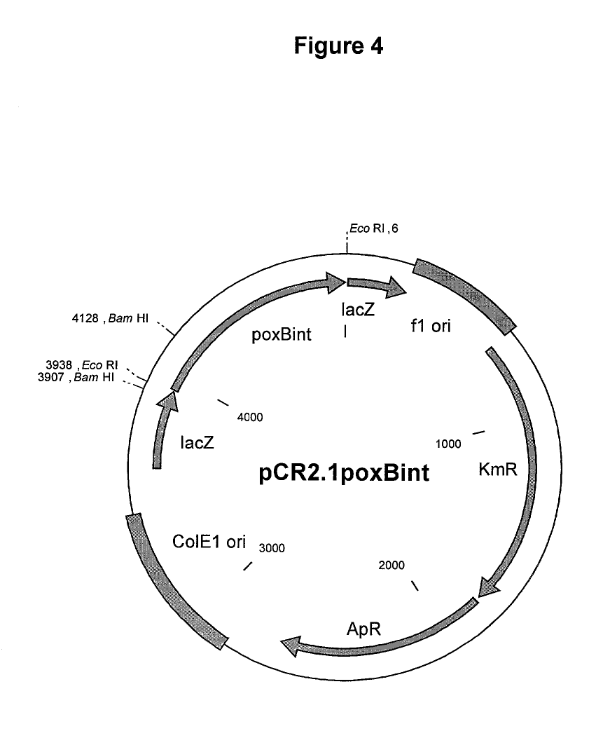

In FIG. 4:

| | |
|---|---|
| ColE1 ori: | Replication origin of the plasmid ColE1 |
| lacZ: | Cloning relict of the lacZα gene fragment |
| fl ori: | Replication origin of phage fl |
| KmR: | Kanamycin resistance |
| ApR: | Ampicillin resistance |
| poxBint: | internal fragment of the poxB gene |

The following abbreviations have also been used herein:

| | |
|---|---|
| AccI: | Cleavage site of the restriction enzyme AccI |
| BamHI: | Cleavage site of the restriction enzyme BamHI |
| EcoRI: | Cleavage site of the restriction enzyme EcoRI |
| HindIII: | Cleavage site of the restriction enzyme HindIII |
| KpnI: | Cleavage site of the restriction enzyme KpnI |
| PstI: | Cleavage site of the restriction enzyme PstI |
| PvuI: | Cleavage site of the restriction enzyme PvuI |
| SalI: | Cleavage site of the restriction enzyme SalI |
| SacI: | Cleavage site of the restriction enzyme SacI |
| SmaI: | Cleavage site of the restriction enzyme SmaI |
| SphI: | Cleavage site of the restriction enzyme SphI |
| XbaI: | Cleavage site of the restriction enzyme XbaI |
| XhoI: | Cleavage site of the restriction enzyme XhoI |

The following examples will further illustrate this invention. The molecular biology techniques, e.g. plasmid DNA isolation, restriction enzyme treatment, ligations, standard transformations of Escherichia coli etc. used are, (unless stated otherwise), are described by Sambrook et al., (Molecular Cloning. A Laboratory Manual (1989) Cold Spring Harbor Laboratories, USA).

### EXAMPLE 1

### Construction of a Gene Library of Corynebacterium glutamicum Strain ASO019

A DNA library of Corynebacteruim glutamicum strain AS019 (Yoshihama et al., Journal of Bacteriology 162, 591-597 (1985)) was constructed using λZap Express™ system, (Short et al., (1988) Nucleic Acids Research 16: 7583-7600), as described by O'Donohue (O'Donohue, M. (1997). The Cloning and Molecular Analysis of Four Common Aromatic Amino Acid Biosynthetic Genes from Corynebacterium glutamicum. Ph.D. Thesis, National University of Ireland, Galway). λZap Express™ kit was purchased from Stratagene (Stratagene, 11011 North Torrey Pines Rd., La Jolla, Calif. 92037) and used according to the manufacturer's instructions. AS019-DNA was digested with restriction enzyme Sau3A and ligated to BamHI treated and dephosphorylated λZap Express™ arms.

### EXAMPLE 2:

### Cloning and Sequencing of the gnd Gene 2.1 Construction of a gnd Probe

A radio-labeled oligonucleotide, internal to the gnd gene, was used to probe the AS019 λZap Express™ library described above. The oligonucleotide was produced using degenerate PCR primers internal to the gnd gene. The degenerate nucleotide primers designed for the PCR amplification of gnd DNA fragments were as follows:

gnd1: 5′ ATG GTK CAC ACY GGY ATY GAR TA 3′ (SEQ ID NO 7)

gnd2: 5′ RGT CCA YTT RCC RGT RCC YTT 3′ (SEQ ID NO 8)

with R=A+G; Y=C+T; K=T+G.

The estimated size of the resulting PCR product was 252 bp approximately. Optimal PCR conditions were determined to be as follows:

35 cycles

94° C. for 1 minute

55° C. for 1 minute

72° C. for 30 seconds

2.5-3.5 mM MgCl₂

100-150 ng AS019 genomic DNA.

Sequence analysis of the resulting PCR product confirmed the product to be an internal portion of a gnd gene. Sequence analysis was carried out using the universal forward and reverse primers, and T7 sequencing kit from Pharmacia Biotech, (St. Albans, Herts, UK). The sequence of the PCR product is shown in SEQ ID No. 1.

2.2 Cloning

Screening of the AS019 λZap Express™ library was carried out according to the λZap Express™ system protocol, (Stratagene, 11011 North Torrey Pines Rd., La Jolla, Calif. 92037). Southern Blot analysis was then carried out on isolated clones. Southern transfer of DNA was as described in the Schleicher and Schuell protocols manual employing Nytran™ as membrane (“Nytran, Modified Nylon-66 Membrane Filters” (March 1987), Schleicher and Schuell, Dassel, Germany). Double stranded DNA fragments, generated using the same primers and optimal PCR conditions as described above, were radio-labeled with α-³²P-dCTP using the Multiprime™ DNA labeling kit from Amersham Life Science (Amersham Pharmacia Biotech UK Limited, Little Chalfont, Buckinghamshire, UK) according to the manufacturers instructions. Prehybridization, hybridization and washing conditions were as described in the Schleicher and Schuell protocols manual. Autoradiography was carried out according to the procedure outlined in the handbook of Sambrook et al. using AgFa Curix RPIL film. Thus several gnd clones were identified. Plasmid DNA was isolated from one of the clones, designated pBGNA (FIG. 3) and chosen for further analysis.

2.3 Sequencing

The Sanger Dideoxy chain termination method of Sanger et al. (Proceedings of the National Academy of Sciences USA 74, 5463-5467 (1977)) was used to sequence the cloned insert of pBGNA. The method was applied using the T7 sequencing kit and α-³⁵S-dCTP from Pharmacia Biotech (St. Albans, Herts, UK). Samples were electrophoresed for 3-8 hours on 6% polyacrylamide/urea gels in TBE buffer at a constant current of 50 mA, according to the Pharmacia cloning and sequencing instructions manual (“^{T7} Sequencing™Kit”,ref.XY-010-00-19, Pharmacia Biotech, 1994). Sequence analysis was carried out using internal primers designed from the sequence known of the internal gnd PCR product (SEQ ID NO 1) allowing the entire gnd gene sequence to be deduced. The sequences of the internal primers were as follows:

Internal primer 1: 5′ GGT GGA TGC TGA AAC CG 3′ (SEQ ID NO 9)

Internal primer 2: 5′ GCT GCA TGC CTG CTG CG 3′ (SEQ ID NO 10)

Internal primer 3: 5′ TTG TTG CTT ACG CAC AG 3′ (SEQ ID NO 11)

Internal primer 4: 5′ TCG TAG GAC TTT GCT GG 3′ (SEQ ID NO 12)

Sequences obtained were analyzed using the DNA Strider program, (Marck (1988), Nucleic Acids Research 16: 1829-1836), version 1.0 on an Apple Macintosh computer. This program allowed for analyses such as restriction site usage, open reading frame analysis and codon usage determination. Searches between DNA sequences obtained and those in EMBL and Genbank databases were performed using the BLAST program (Altschul et al., (1997), Nucleic Acids Research 25: 3389-3402). DNA and protein sequences were aligned using the Clustal V and Clustal W programs (Higgins and Sharp, 1988 Gene 73: 237-244).

The sequence thus obtained is shown in SEQ ID NO 2. The analysis of the nucleotide sequence obtained revealed an open reading frame of 1377 base pairs which was designated as gnd gene. It codes for a protein of 459 amino acids shown in SEQ ID NO 3.

### EXAMPLE 3

### Preparation of the Shuttle Vector pEC-T18mob2

The E. coli—C. glutamicum shuttle vector pEC-T18mob2 was constructed according to the prior art. The vector contains the replication region, rep, of the plasmid pGA I including the replication effector, per (US-A-5,175,108; Nesvera et al., Journal of Bacteriology 179, 1525-1532 (1997)), the tetracycline resistance-imparting tetA(Z) gene of the plasmid, pAG1 (US-A-5,158,891; gene library entry at the National Center for Biotechnology Information (NCBI, Bethesda, Md., USA) with accession number AF121000), the replication region, oriV, of the plasmid pMB I (Sutcliffe, Cold Spring Harbor Symposium on Quantitative Biology 43, 77-90 (1979)), the lacZ gene fragment including the lac promoter and a multiple cloning site (mcs) (Norrander et al. Gene 26, 101-106 (1983)) and the mob region of the plasmid RP4 (Simon et al.,(1983) Bio/Technology 1:784-791).

The vector constructed was transformed in the E. coli strain DH5(x (Hanahan, In: DNA cloning. A practical approach. Vol. I. IRL-Press, Oxford, Washington D.C., USA, 1985). Selection for plasmid-carrying cells was made by plating out the transformation batch on LB agar (Sambrook et al., Molecular cloning: a laboratory manual. 2^{nd} Ed. Cold Spring Harbor Laboratory Press, Cold Spring Harbor, N.Y., USA, 1989), which had been supplemented with 5 mg/l tetracycline. Plasmid DNA was isolated from a transformant with the aid of the QlAprep Spin Miniprep Kit from Qiagen and checked by restriction with the restriction enzyme EcoRI and HindIII subsequent agarose gel electrophoresis (0.8%). The plasmid was called pEC-Tl8mob2 and is shown in FIG. 1. It is deposited in the form of the strain Escherichia coli K-12 strain DH5α/pEC-Tl8mob2 at the Deutsche Sammlung fir Mikroorganismen und Zellkulturen (DSMZ=German Collection of Microorganisms and Cell Cultures, Braunschweig, Germany) as DSM 13244.

### EXAMPLE 4

### Cloning of the gnd Gene into the E. coli—C. glutamicum Shuttle Vector pEC-T18mob2

PCR was used to amplify DNA fragments containing the entire gnd gene of C. glutamicum and flanking upstream and downstream regions using pBGNA as template. PCR reactions were carried out using oligonucleotide primers designed from SEQ ID NO 2. The primers used were:

gnd fwd. primer: 5′ ACT CTA GTC GGC CTA AAA TGG 3′ (SEQ ID NO 13)

gnd rev. primer: 5′ CAC ACA GGA AAC AGA TAT GAC 3′ (SEQ ID NO 14).

PCR parameters were as follows:

35 cycles

95° C. for 6 minutes

94° C. for 1 minute

50° C. for 1 minute

72° C. for 45 seconds

1 mM MgC1₂

approx. 150-200ng pBGNA-DNA as template.

The PCR product obtained was cloned into the commercially available pGEM-T vector purchased from Promega Corp. (pGEM-T Easy Vector System 1, cat. no. A1360, Promega UK, Southampton) using E. coli strain JM109 (Yanisch-Perron et al. Gene, 33: 103-119 (1985)) as a host. The entire gnd gene was subsequently isolated from the pGEM T-vector on an EcoRI fragment and cloned into the lacZ EcoRI site of the E. coli—C. glutamicum shuttle vector pEC-T18mob2 (FIG. 1), and designated pECgnd (FIG. 2). Restriction enzyme analysis with AccI (Boehringer Mannheim GmbH, Germany) revealed the correct orientation (i.e., downstream the lac-Promotor) of the gnd gene in the lacZα gene of pEC-T18mob2.

EXAMPLE 5

### Preparation of Amino Acid Producers with Amplified 6-phosphogluconate Dehydrogenase

Plasmid pECgnd from Example 3 was electroporated by the electroporation method of Tauch et al. (FEMS Microbiological Letters, 123:343-347 (1994)) in the strains Corynebacteruim glutamicum DSM 5399 and DSM 5714. The strain DSM 5399 is a threonine producer described in EP-B-0358940. The strain DSM 5714 is a lysine producer described in EP-B-0435132. Selection of transformants was carried out by plating out the electroporation batch on LB agar (Sambrook et al., Molecular cloning: a laboratory manual. 2^{nd} Ed. Cold Spring Harbor Laboratory Press, Cold Spring Harbor, N.Y., 1989), which had been supplemented with 25 mg/l kanamycin. The strains DSM5399/pECgnd and DSM5714/pECgnd were formed in this manner.

### EXAMPLE 6

### Preparation of Threonine

The C. glutamicum strain DSM5399/pECgnd obtained in Example 5 was cultured in a nutrient medium suitable for the production of threonine and the threonine content in the culture supernatant was determined. For this, the strain was first incubated on an agar plate with the corresponding antibiotic (brain-heart agar with tetracycline (5 mg/l)) for 24 hours at 33° C. Starting from this agar plate culture, a preculture was seeded (10 ml medium in a 100 ml conical flask). Brain-heart broth (Merck, Darmstadt, Germany) was used as the medium for the preculture. Tetracycline (5 mg/ l) was added to this medium. The preculture was incubated for 24 hours at 33° C. at 240 rpm on a shaking machine. A main culture was seeded from this preculture such that the initial OD (660 nm) of the main culture was 0.1. The medium MM-threonine was used for the main culture.

| | Medium MM-threonine | |
|---|---|---|
| CSL | 5 | g/l |
| MOPS | 20 | g/l |
| Glucose(autoclaved separately) | 50 | g/l |
| Salts: | | |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| CaCO₃ | 25 | g/l |

The CSL (corn steep liquor), MOPS (morpholinopropanesulfonic acid) and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) was added. Culturing was carried out at 33° C. and 80% atmospheric humidity. After 48 hours, the OD was determined at a measurement wavelength of 660 nm with a Biomek 1000 (Beckmann Instruments GmbH, Munich). The concentration of threonine formed was determined with an amino acid analyzer from Eppendorf-BioTronik (Hamburg, Germany) by ion exchange chromatography and post-column derivation with ninhydrin detection. The result of the experiment is shown in Table 1.

TABLE 1

| | OD | L-Threonin |
|---|---|---|
| Strain | (660 nm) | g/l |
| DSM5399/pECgnd | 11.9 | 1.29 |
| DSM5399 | 11.8 | 0.33 |

### EXAMPLE 7

### Preparation of Lysine

The C. glutamicum strain DSM5714/pECgnd obtained in Example 5 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l)) for 24 hours at 33° C. Starting from this agar plate culture, a preculture was seeded (10 ml medium in a 100 ml conical flask). The complete medium Cg III was used as the medium for the preculture.

| | Medium Cg III | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |

The pH was brought to pH 7.4

Tetracycline (5 mg/l) was added to this medium. The preculture was incubated for 24 hours at 33° C. at 240 rpm on a shaking machine. A main culture was seeded from this preculture such that the initial OD (660nm) of the main culture was 0.05. Medium MM was used for the main culture.

| | Medium MM | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| (NH₄)₂SO₄ | | |
| KH₂PO₄ | 25 | g/l |
| MgSO₄ * 7 H₂O | 0.1 | g/l |
| CaCl₂ * 2 H₂O | 1.0 | g/l |
| FeSO₄ * 7 H₂O | 10 | mg/l |
| MnSO₄ * H₂O | 10 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing was carried out in a 10 ml volume in a 100 ml conical flask with baffles. Tetracycline (5 mg/l) was added. Culturing was carried out at 33° C. and 80% atmospheric humidity.

After 48 hours, the OD was determined at a measurement wavelength of 660 nm with a Biomek 1000 (Beckmann Instruments GmbH, Mu inchen). The amount of lysine formed was determined with an amino acid analyzer from Eppendorf-BioTronik (Hamburg, Germany) by ion exchange chromatography and post-column derivation with ninhydrin detection. The result of the experiment is shown in Table 2.

TABLE 2

| | OD | Lysine HCl |
|---|---|---|
| Strain | (660 nm) | g/l |
| DSM5715/pECgnd | 7.7 | 14.7 |
| DSM5715 | 7.1 | 13.7 |

### EXAMPLE 8

### Preparation of a Genomic Cosmid Gene Library from Corynebacteruim glutamicum ATCC 13032

Chromosomal DNA from Corynebacteruim glutamicum ATCC 13032 was isolated as described by Tauch et al., (1995, Plasmid 33:168-179), and partly cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Molecular Biochemicals, Mannheim, Germany, Product Description SAP, Code no. 1758250). The DNA of the cosmid vector SuperCosl (Wahl et al. (1987) Proceedings of the National Academy of Sciences USA 84:2160-2164), obtained from Stratagene (La Jolla, USA, Product Description SuperCosl Cosmid Vektor Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA was then cleaved with the restriction enzyme BamHi (Amersham Pharmacia, Freiburg, Germany, Product Description BamHI, Code no. 27-0868-04). The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no.27-0870-04). The ligation mixture was then packed in phages with the aid of Gigapack II XL Packing Extracts (Stratagene, La Jolla, USA, Product Description Gigapack II XL Packing Extract, Code no. 200217). For infection of the E. coli strain NM554 (Raleigh et al. 1988, Nucleic Acid Research 16:1563-1575) the cells were taken up in 10 mM MgSO₄ and mixed with an aliquot of the phage suspension. The infection and titering of the cosmid library were carried out as described by Sambrook et al. (1989, Molecular Cloning: A laboratory Manual, Cold Spring Harbor), the cells being plated out on LB agar (Lennox, 1955, Virology 1:190)+100 μg/ml ampicillin. After incubation overnight at 37° C., recombinant individual clones were selected.

### EXAMPLE 9

### Isolation and Sequencing of the poxB Gene

The cosmid DNA of an individual colony (Example 8) was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany). The DNA of the sequencing vector pZero-1, obtained from Invitrogen (Groningen, Holland, Product Description Zero Background Cloning Kit, Product No. K2500-01) was cleaved with the restriction enzyme BamHI (Amersham Pharmacia, Freiburg, Germany, Product Description BamHI, Product No. 27-0868-04).

The ligation of the cosmid fragments in the sequencing vector pZero-1 was carried out as described by Sambrook et al. (1989, Molecular Cloning: A laboratory Manual, Cold Spring Harbor), the DNA mixture being incubated overnight with T4 ligase (Pharmacia Biotech, Freiburg, Germany). This ligation mixture was then electroporated (Tauch et al. 1994, FEMS Microbiol Letters, 123:343-7) into the E. coli strain DH5α cMCR (Grant, 1990, Proceedings of the National Academy of Sciences U.S.A., 87:4645-4649) and plated out on LB agar (Lennox, 1955, Virology, 1:190) with 50 μg/ml zeocin. The plasmid preparation of the recombinant clones was carried out with Biorobot 9600 (Product No. 900200, Qiagen, Hilden, Germany). The sequencing was carried out by the dideoxy chain-stopping method of Sanger et al. (1977, Proceedings of the National Academies of Sciences U.S.A., 74:5463-5467) with modifications according to Zimmermann et al. (1990, Nucleic Acids Research, 18:1067). The “RR dRhodamin Terminator Cycle Sequencing Kit” from PE Applied Biosystems(Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231) version 97-0. The individual sequences of the pZerol derivatives were assembled to a continuous contig. The computer-assisted coding region analysis were prepared with the XNIP program (Staden, 1986, Nucleic Acids Research 14:217-231). Further analyses were carried out with the “BLAST search program” (Altschul et al., 1997, Nucleic Acids Research 25:3389-3402), against the non-redundant databank of the “National Center for Biotechnology Information” (NCBI, Bethesda, Md., USA).

The resulting nucleotide sequence is shown in SEQ ID No. 4. Analysis of the nucleotide sequence showed an open reading frame of 1737 base pairs, which was called the poxB gene. The poxB gene codes for a polypeptide of 579 amino acids (SEQ ID NO. 5).

### EXAMPLE 10

### Preparation of an Integration Vector for Integration Mutagenesis of the poxB Gene

From the strain ATCC 13032, chromosomal DNA was isolated by the method of Eikmanns et al. (Microbiology 140: 1817-1828 (1994)). On the basis of the sequence of the poxB gene known for C. glutamicum from Example 9, the following oligonucleotides were chosen for the polymerase chain reaction:

poxBint1 (SEQ ID NO 15): 5′ TGC GAG ATG GTG AAT GGT GG 3′

poxBint2 (SEQ ID NO 16): 5′ GCA TGA GGC AAC GCA TTA GC 3′

The primers shown were synthesized by MWG Biotech (Ebersberg, Germany) and the PCR reaction was carried out by the standard PCR method of Innis et al. (PCR protocols. A guide to methods and applications, 1990, Academic Press) with Pwo-Polymerase from Boehringer. With the aid of the polymerase chain reaction, a DNA fragment approx. 0.9 kb in size was isolated, this carrying an internal fragment of the poxB gene and being shown in SEQ ID No:6.

The amplified DNA fragment was ligated with the TOPO TA Cloning Kit from Invitrogen Corporation (Carlsbad, Calif., USA; Catalogue Number K4500-01) in the vector pCR2.1-TOPO (Mead at al. (1991) Bio/Technology 9:657-663). The E. coli Stamm DH5α was then electroporated with the ligation batch (Hanahan, In: DNA cloning. A practical approach. Vol. I. IRL-Press, Oxford, Washington D.C., USA, 1985). Selection for plasmid-carrying cells was made by plating out the transformation batch on LB agar (Sambrook et al., Molecular cloning: a laboratory manual. 2^{nd} Ed. Cold Spring Harbor Laboratory Press, Cold Spring Harbor, N.Y., 1989), which had been supplemented with 25 mg/l kanamycin. Plasmid DNA was isolated from a transformant with the aid of the QIAprep Spin Miniprep Kit from Qiagen and checked by restriction with the restriction enzyme EcoRI and subsequent agarose gel electrophoresis (0.8%). The plasmid was called pCR2.1poxBint (FIG. 4).

Plasmid pCR2.1 poxBint has been deposited in the form of the strain Escherichia coli DH5α/pCR2. lpoxBint as DSM 13114 at the Deutsche Sammlung f dir Mikroorganismen und Zellkulturen (DSMZ=German Collection of Microorganisms and Cell Cultures, Braunschweig, Germany) in accordance with the Budapest Treaty.

### EXAMPLE 11

### Integration Mutagenesis of the poxB Gene in the Lysine Producer DSM 5715

The vector pCR2.1poxBint mentioned in Example 10 was electroporated by the electroporation method of Tauch et al.(FEMS Microbiological Letters, 123:343-347 (1994)) in Corynebacteruim glutamicum DSM 5715. Strain DSM 5715 is an AEC-resistant lysine producer. The vector pCR2.1poxBint cannot replicate independently in DSM5715 and is retained only if it has integrated into the cell's chromosome. Selection of clones with pCR2.1poxBint integrated into the chromosome was carried out by plating out the electroporation batch on LB agar (Sambrook et al., Molecular Cloning: A Laboratory Manual. 2^{nd} ed., Cold Spring Harbor Laboratory Press, Cold Spring Harbor, N.Y.), which had been supplemented with 15 mg/l kanamycin. For detection of the integration, the poxBint fragment was labeled with the Dig hybridization kit from Boehringer by the method of “The DIG System Users Guide for Filter Hybridization” of Boehringer Mannheim GmbH (Mannheim, Germany, 1993). Chromosomal DNA of a potential integrant was isolated by the method of Eikmanns et al. (Microbiology 140: 1817-1828 (1994)) and in each case cleaved with the restriction enzymes SalI, SacI and HindIII. The fragments formed were separated by agarose gel electrophoresis and hybridized at 68° C. with the Dig hybridization kit from Boehringer. The plasmid pCR2.1poxBint mentioned in Example 9 had been inserted into the chromosome of DSM5715 within the chromosomal poxB gene. The strain was called DSM5715::pCR2.1poxBint.

### EXAMPLE 12

### Effect of Over-expression of the gnd Gene with Simultaneous Elimination of the poxB Gene on the Preparation of Lysine

12.1 Preparation of the Strain DSM5715::pCR2.1poxBint/pECgnd

The strain DSM5715::pCR2.1poxBint was transformed with the plasmid pECgnd using the electroporation method described by Liebl et al., (FEMS Microbiology Letters, 53:299-303 (1989)). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 5 mg/l tetracycline and 25 mg/l kanamycin. Incubation was carried out for 2 days at 33° C.

Plasmid DNA was isolated in each case from a transformant by conventional methods (Peters-Wendisch et al., 1998, Microbiology 144, 915-927), cleaved with the restriction endonuclease AccI, and the plasmid was checked by subsequent agarose gel electrophoresis. The strain obtained in this way was called DSM5715 :pCR2.1poxBint/pECgnd.

12.2 Preparation of L-lysine

The C. glutamicum strain DSM5715::pCR2.1poxBint/pECgnd obtained in Example 12.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l) and kanamycin (25 mg/l)) for 24 hours at 33° C. The cultures of the comparison strains were supplemented according to their resistance to antibiotics. Starting from this agar plate culture, a preculture was seeded (10 ml medium in a 100 ml conical flask). The complete medium CgIII was used as the medium for the preculture.

| | Medium Cg III | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |

The pH was brought to pH 7.4

Tetracycline (5 mg/l) and kanamycin (25 mg/l) were added to this. The preculture was incubated for 16 hours at 33° C. at 240 rpm on a shaking machine. A main culture was seeded from this preculture such that the initial OD (660nm) of the main culture was 0.1. Medium MM was used for the main culture.

| | Medium MM | |
|---|---|---|
| CSL (corn steep liquor) | 5 | g/l |
| MOPS (morpholinopropanesulfonic acid) | 20 | g/l |
| Glucose (autoclaved separately) | 58 | g/l |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| L-Leucine (sterile-filtered) | 0.1 | g/l |
| CaCO₃ | 25 | g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing was carried out in a 10 ml volume in a 100 ml conical flask with baffles. Tetracycline (5 mg/l) and kanamycin (25 mg/l) were added. Culturing was carried out at 33° C. and 80% atmospheric humidity.

After 72 hours, the OD was determined at a measurement wavelength of 660 nm with a Biomek 1000 (Beckmann Instruments GmbH, Munchen). The amount of lysine formed was determined with an amino acid analyzer from Eppendorf-BioTronik (Hamburg, Germany) by ion exchange chromatography and post-column derivatization with ninhydrin detection. The result of the experiment is shown in Table 3.

TABLE 3

| | OD | L-Lysine HCl |
|---|---|---|
| Strain | (660 nm) | g/l |
| DSM5715 | 10.8 | 16.0 |
| DSM5715/pECgnd | 7.6 | 16.5 |
| DSM5715::pCR2.1poxBint | 7.1 | 16.7 |
| DSM5715::pCR2.1poxBint/ | 7.2 | 17.1 |
| pECgnd | | |

What is claimed is:

1. A process for the preparation of L-lysine, comprising:
a) fermenting an L-lysine producing coryneform bacteria in a culture medium, the bacteria having at least an overexpressed gene encoding 6-phosphogluconate dehydrogenase;
b) concentrating L-lysine produced by said fermenting in the culture medium or in the cells of the bacteria; and
c) isolating the L-lysine produced;
wherein intracellular activity of pyruvate oxidase encoded by a pyruvate oxidase gene is decreased or switched off in the bacteria.

2. The process according to claim 1, wherein an endogenous gene encoding 6-phosphogluconate dehydrogenase is used as the overexpressed gene encoding 6-phosphogluconate dehydrogenase.

3. The process according to claim 1, wherein the overexpressed gene encoding 6-phosphogluconate dehydrogenase is produced by transforming the bacteria with a plasmid vector carrying at least a gene encoding 6-phosphogluconate dehydrogenase and a promoter.

4. The process according to claim 1, wherein the bacteria is a strain of the genus Corynebacterium.

5. A process for the preparation of an L-amino acid, comprising:
a) fermenting an L-amino acid producing coryneform bacteria in a culture medium, the bacteria having at least an overexpressed gnd gene encoding 6-phosphogluconate dehydrogenase;
b) concentrating L-amino acid produced by said fermenting in the culture medium or in the cells of the bacteria; and
d) isolating the L-amino acid produced;
wherein intracellular activity of pyruvate oxidase encoded by a pyruvate oxidase gene is decreased or switched off in the bacteria; and
wherein the L-amino acid is selected from the group consisting of L-threonine, L-isoleucine and L-tryptophan.

6. An L-lysine producing coryneform microorganism having increased intracellular activity of 6-phosphogluconate dehydrogenase and decreased intracellular activity of pyruvate oxidase.

7. The plasmid vector pEC-T18mob2 deposited under the designation DSM 13244 in E. coli K-12 DH5.

8. A coryneform microorganism transformed by introduction of the plasmid vector of claim 7, the coryneform microorganism also having a gene encoding 6-phosphogluconate dehydrogenase.

9. The coryneform microorganism of claim 8, wherein the coryneform microorganism is of the genus Corynebacterium.
